(19) United States
(12) Patent Application Publication
(10) Pub. No.: US 2018/0139528 A1
(43) Pub. Date: May 17, 2018

(54) Sensible Wired Earphones

(71) Applicant: Chi-Cheng WEN, TAOYUAN CITY (TW)

(72) Inventor: Chi-Cheng WEN, TAOYUAN CITY (TW)

(21) Appl. No.: 15/355,034

(22) Filed: Nov. 17, 2016

(51) Int. Cl. H04R 1/10; G06F 3/16; A61B 5/00; A61B 5/04; A61B 5/0205; A61B 5/1455; A61B 5/11

(52) U.S. Cl. CPC H04R 1/1041; G06F 3/165; H04R 1/1016; A61B 5/6803; A61B 5/04012; A61B 5/0205; A61B 5/021; A61B 5/14552; A61B 5/1123; A61B 5/7405; A61B 5/746; H04R 2201/107; A61B 5/02055

(57) ABSTRACT

Sensible wired earphones are electrically connected with information equipment and contain: at least one body, a controller, and a telecommunication connector. Each of the at least one body includes a sensing unit, a micro-control unit, a casing, and a speaker. The sensing unit is configured to acquire user's photoplethysmography (PPG) signals, the micro-control unit is electrically connected with said each body, the sensing unit, and the telecommunication connector which is electrically coupled with the information equipment. The micro-control unit is configured to process and analyze the user's PPG signals, and the user's PPG signals are processed and analyzed by the information equipment. The controller includes a controlling interface electrically connected with the micro-control unit, the controller is electrically in connection with the telecommunication connector via a first electric wire, and said each body is electrically connected with the micro-control unit via a second electric wire.

### FIELD OF THE INVENTION

The present invention relates to sensible wired earphones which track, record and manage user's physiological state and play the music simultaneously.

### BACKGROUND OF THE INVENTION

A wearable device (such as smart watch or smart bracelet) contains a biological information sensor configured to sense user's biological information inclusive of user's pulse and temperature, and software saved in information equipment and connecting with the smart watch or the smart bracelet processes and analyzes biological information so as to obtain user's biological data, such as user's heart rate, temperature, blood oxygen concentration (SpO2), blood pressure, and heart rate variability (HRV).

Preferably, the user's biological data is tracked, recorded and managed by an application program set in the information equipment. Wired earphones sends signals sensed by a sensor to a processor in a wireless transmission manner (such as bluetooth transmission), but such wired earphones are complicated and have high production cost.

Furthermore, the sensor of the conventional wearable device is interfered easily to cause sensing error.

The present invention has arisen to mitigate and/or obviate the afore-described disadvantages.

### SUMMARY OF THE INVENTION

The primary objective of the present invention is to provide sensible wired earphones which track, record and manage user's physiological state and play the music simultaneously

To obtain above-mentioned objective, sensible wired earphones provided by the present invention are electrically connected with information equipment and contains: at least one body, a controller, and a telecommunication connector, wherein each of the at least one body includes a sensing unit, a micro-control unit, a casing, and a speaker accommodated in the casing.

The sensing unit is configured to acquire user's photoplethysmography (PPG) signals.

The micro-control unit is electrically connected with said each body, the sensing unit, and the telecommunication connector, and the telecommunication connector is electrically coupled with the information equipment so as to obtain power from the information equipment, to exchange data with the information equipment, to send the data from the information equipment, and to drive said each body to play music.

The micro-control unit is configured to process and analyze the user's PPG signals, and the user's PPG signals are processed and analyzed by the information equipment, thus obtaining user's biological information.

The controller includes a controlling interface electrically connected with the micro-control unit so that user starts or stops media playing of the information equipment and controls the sensing unit.

The controller is electrically in connection with the telecommunication connector via a first electric wire, and said each body is electrically connected with the micro-control unit via a second electric wire.

Preferably, said each body includes a first metallic part, and the sensible wired earphones further contains a second metallic part, wherein the first metallic part is fixed on and exposes outside said each body, and the second metallic part is mounted on and exposes outside the controller, wherein the micro-control unit is configured to process user's electrocardiogram (ECG) signals sensed by the first metallic part and the second metallic part, the ECG signals are processed and analyzed by the micro-control unit and/or the information equipment so as to acquires the user's biological information, wherein the controller is electrically in connection with the telecommunication connector via the first electric wire, and said each body is electrically connected with the micro-control unit via the second electric wire, wherein the second metallic part contacts with user's skin so as to measure user's electrocardiogram (ECG).

Preferably the user's biological information is any one or any combination of any two or more of heart rate, user's temperature, blood oxygen concentration (SpO2), blood pressure, heart rate variability (HRV), and motion status (static/dynamic status).

Preferably, the sensing unit includes a photoplethysmography (PPG) sensor and an analog front-end (AFE) chip; the PPG sensor is mounted on one side of said each body facing the user so as to acquire user's photoplethysmography (PPG) signals, and the AFE chip is employed to collect and convert the user's ECG signals and the user's PPG signals into the data so that the data is processed and analyzed by the micro-control unit.

Preferably, the AFE chip is integrated with the micro-control unit, and the AFE chip and the micro-control unit are accommodated in said body.

Preferably, the AFE chip is integrated with the micro-control unit, and the AFE chip and the micro-control unit are accommodated in the controller.

Preferably, said each body is an in-ear earphone or a headphone.

Preferably, said each body further includes a microphone electrically connected with the micro-control unit.

Preferably, the telecommunication connector is any one of USB connector, USB type-C connector, and lightning connector.

Preferably, the micro-control unit includes algorithm set therein so as to analyze the user's PPG signals and the user's ECG signals, thus obtaining the user's biological information

Preferably, the controller further includes a display unit, and the display unit has at least one LED element and a monitor, wherein the micro-control unit sends warning signals to the user via said each body.

Preferably, the warning signals are any one or any combination of any two or more of sounds, voices and lights.

### BRIEF DESCRIPTION OF THE DRAWINGS

FIG. 1 is a perspective view showing the assembly of sensible wired earphones according to a preferred embodiment of the present invention.

FIG. 2 is a block diagram showing the function of the sensible wired earphones according to a preferred embodiment of the present invention.

FIG. 3 is a block diagram showing the function of sensible wired earphones according to another preferred embodiment of the present invention.

FIG. 4 is a block diagram showing the function of sensible wired earphones according to another preferred embodiment of the present invention.

FIG. 5 is a schematic view showing the application of sensible wired earphones according to the preferred embodiment of the present invention.

### DETAILED DESCRIPTION OF THE PREFERRED EMBODIMENTS

Sensible wired earphones according to a preferred embodiment of the present invention are electrically connected with information equipment D and are capable of playing music, sensing user's biological information, and exchanging data with the information equipment D.

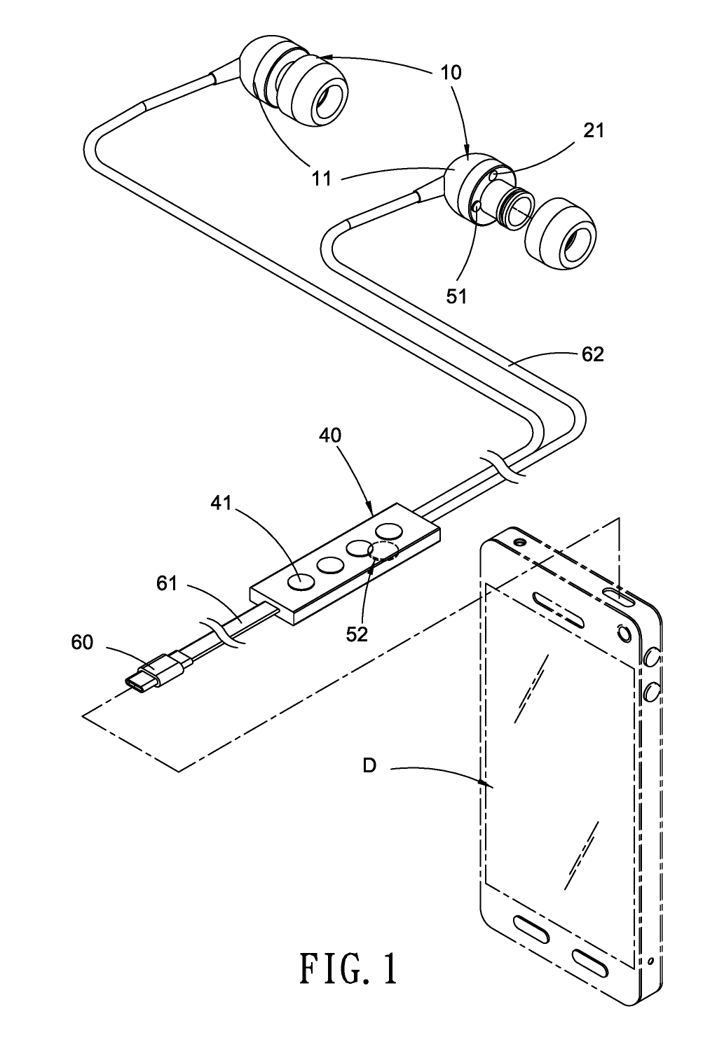

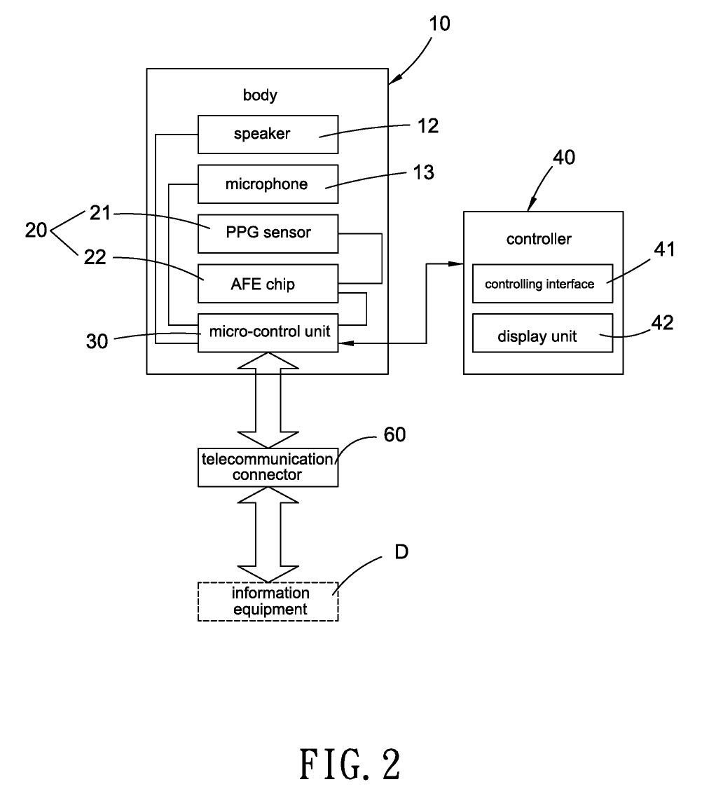

With reference to FIGS. 1 and 2, the sensible wired earphones comprise at least one body 10, each of the at least one body 10 includes a sensing unit 20 and a micro-control unit 30, and the sensible wired earphones further comprise a controller 40 and a telecommunication connector 60.

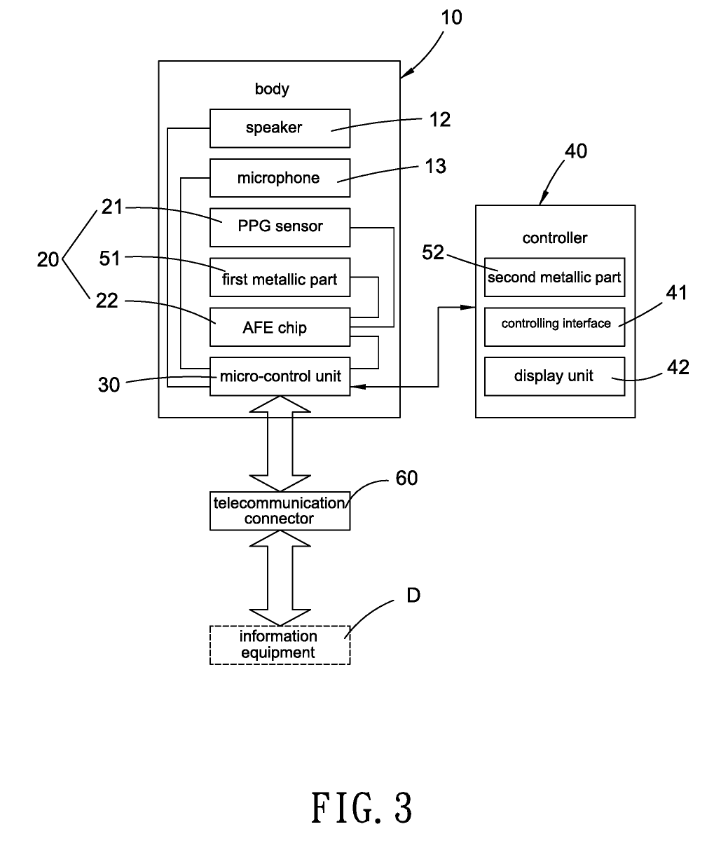

Referring to FIG. 3, said each body 10 includes a first metallic part 51, and the sensible wired earphones further comprises a second metallic part 52, wherein the first metallic part 51 and the second metallic part 52 are applied to sense user's electrocardiogram (ECG) signals.

Preferably, said each body 10 is an in-ear earphone or a headphone.

Said each body 10 also includes a casing 11 and a speaker 12 accommodated in the casing 11 and configured to play the music or to output sound signals.

Said each body 10 further includes a microphone 13 electrically connected with the micro-control unit 30 so as to output voices or the sound signals. The information equipment D outputs the voices and the sound signals, and the voices and the sound signals are sent to and are played by the speaker 12 of said each body 10 via the micro-control unit 30. Preferably, the microphone 13 receives and transmits the voices and the sound signals to the information equipment D via the micro-control unit 30.

The sensing unit 20 includes a photoplethysmography (PPG) sensor 21 and an analog front-end (AFE) chip 22, wherein the PPG sensor 21 is mounted on one side of said each body 10 facing user so as to acquire user's photoplethysmography (PPG) signals, and the AFE chip 22 is employed to collect and convert the user's ECG signals and the user's PPG signals into data so that the data is processed and analyzed by the micro-control unit 30.

The micro-control unit 30 exchanges the data with the information equipment D and transmits the data. Preferably, the micro-control unit 30 controls said each body 10 and the microphone 13 so as to acquire the user's ECG signals via the first metallic part 51 and the second metallic part 52.

The controller 40 includes a controlling interface 41 electrically connected with the micro-control unit 30 so that the user starts or stops media playing (such as the music) of the information equipment D, controls volume, and selects songs by way of the controlling interface 41. In addition, the user is capable of controlling the sensing unit 20 by means of the controlling interface 41. The controlling interface 41 has any one of buttons, touch buttons, and combination of the buttons and the touch buttons. The controller 40 further includes a display unit 42, and the controlling interface 41 and the display unit 42 are electrically in connection with the micro-control unit 30, wherein the display unit 42 has at least one LED element and a monitor (such as LCD monitor) so as to display messages from the micro-control unit 30, and the messages contain any one or any combination of any two or more of power status, warning signals, and heart rate. Preferably, the controller 40 is electrically connected with the information equipment D directly or is electrically connected with the information equipment D via the micro-control unit 30 so as to control the media playing of the information equipment D and to control the sensing unit 20.

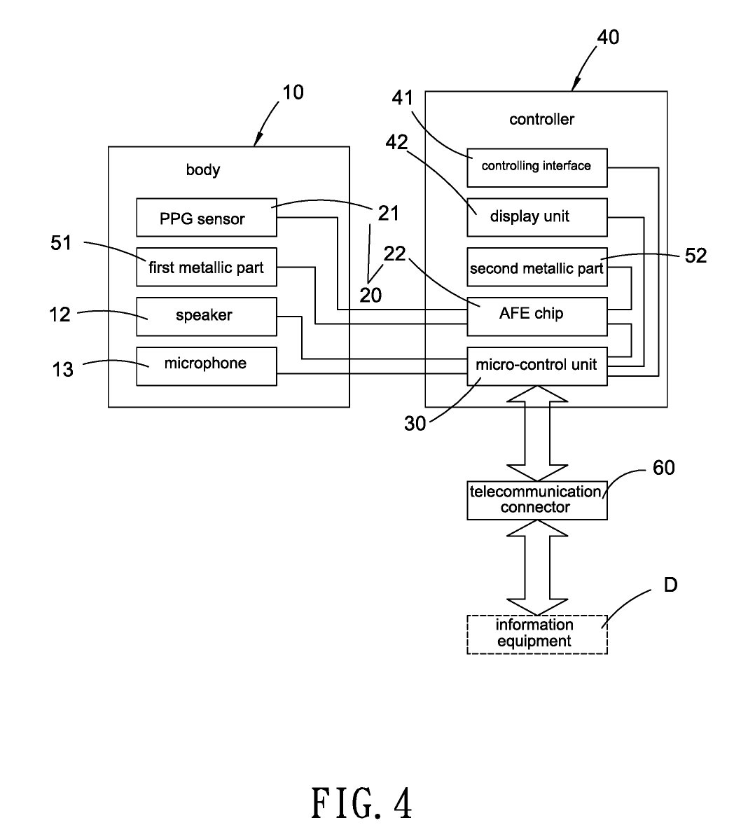

The micro-control unit 30 includes integrated circuit components or is a single chip microcontroller, wherein the AFE chip 22 is integrated with the micro-control unit 30 so as to obtain system on chip (SOC). Preferably, the AFE chip 22 and the micro-control unit 30 are arranged in said each body 10, as shown in FIG. 2. In another embodiment, the AFE chip 22 and the micro-control unit 30 are arranged in the controller 40, as illustrated in FIG. 4.

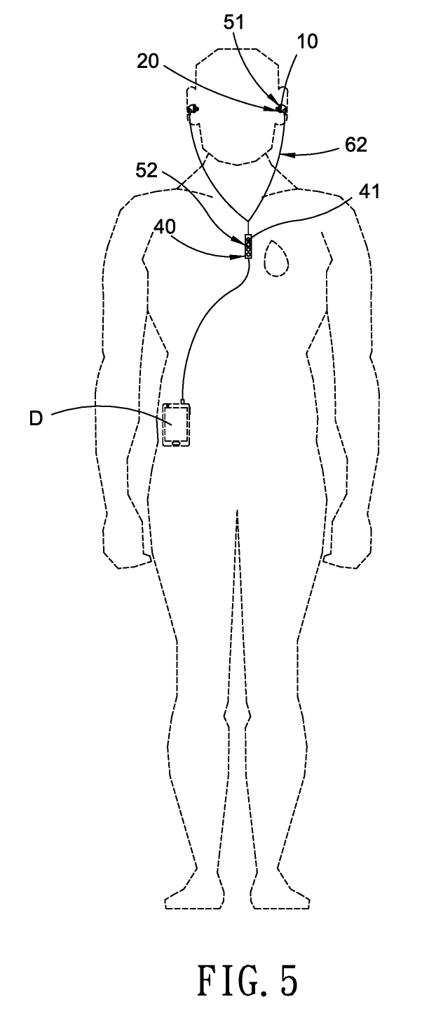

Referring to FIG. 1, the first metallic part 51 is fixed on and exposes outside said each body 10, and the second metallic part 52 is mounted on and exposes outside the controller 40, wherein the controller 40 is electrically in connection with the telecommunication connector 60 via a first electric wire 61, and said each body 10 is electrically connected with the micro-control unit 30 via a second electric wire 62. It is to be noted that the second metallic part 52 contacts with user's skin so as to measure user's electrocardiogram, as shown in FIG. 5, and wherein the sensing unit 20, the first metallic part 51, and the second metallic part 52 are electrically connected with the AFE chip 22 and/or the micro-control unit 30 in a wired connection manner so as to avoid signal interference.

The telecommunication connector 60 is any one of USB connector, USB type-C connector, and lightning connector, and the telecommunication connector 60 is electrically connected with the information equipment D so as to obtain power from the information equipment D, to exchange the data with the information equipment D, and to send the data from the information equipment D.

The micro-control unit 30 is electrically connected with the sensing unit 20, the first metallic unit 51, and the second metallic part 52 so as to process the user's PPG signals and the user's ECG signals. The micro-control unit 30 includes algorithm set therein so as to analyze the user's PPG signals and the user's ECG signals, thus obtaining the user's biological information, such as any one or any combination of any two or more of the heart rate, user's temperature, blood oxygen concentration (SpO2), blood pressure, heart rate variability (HRV), and motion status (static/dynamic status). Preferably, the micro-control unit 30 sends warning signals to the user via said each body 10, and the warning signals are any one or any combination of any two or more of sounds, voices and lights.

In operation, the PPG signals and the ECG signals are processed and analyzed by the micro-control unit 30 and/or the information equipment D, for example, the PPG signals and the ECG signals are transmitted to the information equipment D so that application program (such as APP) or algorithm of the information equipment D acquires the user's biological information, hence the sensible wired earphones track, record and manage user's physiological state and play the music simultaneously.

The information equipment D is electronic equipment configured to process, input, and output various signals and data and to supply power to the sensible wired earphones. The electronic equipment is any one of smart phone, tablet computer, wearable device (such as smart watch or smart bracelet), virtual reality (VR), amplified reality (AR), mixed reality (MR), notebook computer, and personal computer.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. Sensible wired earphones being electrically connected with information equipment and comprising: at least one body, a controller, and a telecommunication connector, wherein each of the at least one body includes a sensing unit, a micro-control unit, a casing, and a speaker accommodated in the casing;
wherein the sensing unit is configured to acquire user's photoplethysmography (PPG) signals;
wherein the micro-control unit is electrically connected with said each body, the sensing unit, and the telecommunication connector, and the telecommunication connector is electrically coupled with the information equipment so as to obtain power from the information equipment, to exchange data with the information equipment, to send the data from the information equipment, and to drive said each body to play music;
wherein the micro-control unit is configured to process and analyze the user's PPG signals, and the user's PPG signals are processed and analyzed by the information equipment, thus obtaining user's biological information;
wherein the controller includes a controlling interface electrically connected with the micro-control unit so that user starts or stops media playing of the information equipment and controls the sensing unit;
wherein the controller is electrically in connection with the telecommunication connector via a first electric wire, and said each body is electrically connected with the micro-control unit via a second electric wire.

2. The sensible wired earphones as claimed in claim 1, wherein said each body includes a first metallic part, and the sensible wired earphones further comprises a second metallic part, wherein the first metallic part is fixed on and exposes outside said each body, and the second metallic part is mounted on and exposes outside the controller, wherein the micro-control unit is configured to process user's electrocardiogram (ECG) signals sensed by the first metallic part and the second metallic part, the ECG signals are processed and analyzed by the micro-control unit and/or the information equipment so as to acquires the user's biological information, wherein the controller is electrically in connection with the telecommunication connector via the first electric wire, and said each body is electrically connected with the micro-control unit via the second electric wire, wherein the second metallic part contacts with user's skin so as to measure user's electrocardiogram (ECG).

3. The sensible wired earphones as claimed in claim 1, wherein the user's biological information is any one or any combination of any two or more of heart rate, user's temperature, blood oxygen concentration (SpO2), blood pressure, heart rate variability (HRV), and motion status (static/dynamic status).

4. The sensible wired earphones as claimed in claim 1, wherein the sensing unit includes a photoplethysmography (PPG) sensor and an analog front-end (AFE) chip; the PPG sensor is mounted on one side of said each body facing the user so as to acquire user's photoplethysmography (PPG) signals, and the AFE chip is employed to collect and convert the user's ECG signals and the user's PPG signals into the data so that the data is processed and analyzed by the micro-control unit.

5. The sensible wired earphones as claimed in claim 4, wherein the AFE chip is integrated with the micro-control unit, and the AFE chip and the micro-control unit are accommodated in said body.

6. The sensible wired earphones as claimed in claim 4, wherein the AFE chip is integrated with the micro-control unit, and the AFE chip and the micro-control unit are accommodated in the controller.

7. The sensible wired earphones as claimed in claim 1, wherein said each body is an in-ear earphone or a headphone.

8. The sensible wired earphones as claimed in claim 1, wherein said each body further includes a microphone electrically connected with the micro-control unit.

9. The sensible wired earphones as claimed in claim 1, wherein the telecommunication connector is any one of USB connector, USB type-C connector, and lightning connector.

10. The sensible wired earphones as claimed in claim 1, Wherein the micro-control unit includes algorithm set therein so as to analyze the user's PPG signals and the user's ECG signals, thus obtaining the user's biological information

11. The sensible wired earphones as claimed in claim 1, wherein the controller further includes a display unit, and the display unit has at least one LED element and a monitor, wherein the micro-control unit sends warning signals to the user via said each body.

12. The sensible wired earphones as claimed in claim 10, wherein the warning signals are any one or any combination of any two or more of sounds, voices and lights.
